(19) United States
(12) Patent Application Publication
(10) Pub. No.: US 2022/0260556 A1
(43) Pub. Date: Aug. 18, 2022

(54) SALIVA TESTING

(71) Applicant: Mint Diagnostics LTD, Sittingbourne (GB)

(72) Inventors: Stefano BORINI, Sittingbourne (GB); Richard WHITE, Sittingbourne (GB); Nikolaos Sotirios VASILAKIS, Sittingbourne (GB)

(21) Appl. No.: 17/624,333

(22) PCT Filed: Jun. 23, 2020
(86) PCT No.: PCT/EP2020/067462

§ 371 Date: Dec. 31, 2021

(30) Foreign Application Priority Data
Jul. 4, 2019 (GB) 1909611.4

(51) Int. Cl. G01N 33/543; A61B 10/00; B01L 3/00

(52) U.S. Cl. CPC G01N 33/543; A61B 10/0051; B01L 2300/0645; B01L 2200/027; B01L 3/5027

(57) ABSTRACT

A saliva test system for performing an ELISA or ELONA test comprises: a narrowing, such as a constriction, of an inlet channel, the narrowing for limiting a volume of collected saliva; and a vent hole to assist flow of the collected saliva through the saliva receiver toward an incubation chamber, the vent hole coupled downstream of the narrow-ing and/or the incubation chamber and openable to enable flow of the collected saliva through the narrowing toward the incubation chamber.

### FIELD OF THE INVENTION

The present invention generally relates to a saliva test system and a method for performing an assay using such a system. Such a system or method may test the saliva for the presence or concentration of at least one analyte, where the analyte is a biomarker or a drug of abuse.

### BACKGROUND TO THE INVENTION

Recent years have seen growth in activity-tracking wearable devices and devices capable of monitoring biometrics of an individual such as heart rate. The direct monitoring of a person's biochemistry is also desirable. Hormones regulate many processes within the body including metabolism, digestion, reproduction, our ability to access energy reserves, our mood and emotions, sleep to name a few. Understanding our endocrinology can help us optimise our health, wellness and/or fitness. For example, cortisol is a stress hormone that is generally released in the human body as a result of physical or psychological stressors. Where the stress response is dysregulated, this can lead to broad health problems and/or performance deterioration, with regard to a person's mental or physical state. Hence it may be desirable to directly monitor cortisol.

Diagnostic tests available for home-use are often qualitative in nature (yes/no). This applies for example to lateral flow home pregnancy tests, and semi-qualitative tests such as lateral flow ovulation tests. For measurement of certain analytes, such as biomarkers or drugs of abuse in the body and subsequent tracking over time, a quantitative test is preferred. However, to obtain reliable quantitative results from analysis of saliva using existing methods, a sample is generally prepared and controlled prior to analysis. For example, to carry out an ELISA (Enzyme-Linked Immunosorbent Assay) test for detecting cortisol concentration of saliva, the sample is centrifuged and the pH checked before analysis. Similarly, for a salivary lateral flow test, the saliva is diluted in buffer solutions before analysis.

Such existing methods are unsuitable for home saliva testing. Ideally, biosensor devices should be suitable for use by an untrained consumer in their own home. A requirement for multiple reagents and/or sample pre-treatment, which generally add complexity to the testing protocol and may introduce significant sources of error, are example barriers to home diagnostic tests becoming more prevalent.

A further barrier is that, to obtain reliable quantitative results from analysis of saliva using existing methods, relatively large volumes of saliva need to be collected. Saliva collection methods such as passive drooling are required with long collection times, e.g., 15 minutes.

The field of saliva testing therefore needs an improved method or device to monitor the levels of target analytes such as a chosen biomarker(s) in an individual's saliva, to allow, e.g., quantitative measurement, greater convenience for the user, speed, accuracy, sensitivity and/or reliability, and preferably without any requirement of a lab environment, trained professionals and/or sample pre-treatment. It is desirable that the testing needs minimum (preferably no) user intervention or input at any stage after the saliva sample collection and during saliva analysis.

For use in understanding the present invention, the following disclosures are referred to:
WO2017/132565 A1: “Saliva Glucose Measurement Devices and Methods” (Labelle et al; published 3.8.17);
US2017/0226557 A1: “Strip-based Electrochemical sensors for Quantitative Analysis of Analytes” (Wang et al; published 10.8.17); and
US2009/0306543 A1: “Specimen Sample Collection Device and Test System” (Slowey et al, published 10.12.09);
U.S. Pat. No. 6,248,598 B1: “Immunoassay that provides for both Collection of Saliva and Assay of Saliva for one or more Analytes with Visual Readout” (Bogema; published 19.6.01)
U.S. Pat. No. 9,223,855 B1: “Method and System for Training Athletes based on Athletic Signatures and a Classification thereof” (Wagner; published 29.12.15);
US20100206748 A1: “Stress Measurement Kit and Stress Measurement Method” (Morita et al; published 19.8.10);
WO2011030093 A1: “Glucose Measurement Method and System” (McColl et al; published 17.3.11);
US2007015286 A1: “Diagnostic Strip Coding System and Related Methods of use” (Neel et al; published 18.1.07);
US2011174616 A1: “Methods for Measuring Physiological Fluids” (Roberts et al; 21.7.11);
US2016313313 A1: “Lateral Flow Assay Apparatus and Method, and Sensor Therefor” (Love et al; published 27.10.16);
JPWO2014181753 A1: “Measuring Device” (Shigeru; published 13.11.14);
US2011108440 A1: “Underfill Recognition System for a Biosensor” (Wu et al; published 12.5.11);
“The lab-on-PCB approach: tackling the μTAS commercial upscaling bottleneck” (Moschou et al; Lab Chip, 2017, 17, 1388. DOI: 10.1039/c71c00121e);
“ELISA-type assays of trace biomarkers using microfluidic methods” (Dong et al; WIREs Nanomed Nanobiotechno. 2017, 9:e1457. DOI: 10.1002/wnan.1457);
“Materials for Microfluidic Immunoassays: A Review” (Mou et al; Adv. Healthcare Mater. 2017, 6, 1601403. DOI: 10.1002/adhm.201601403);
“A Novel Microfluidic Point-of-Care Biosensor System on Printed Circuit Board for Cytokine Detection” (Evans et al; Sensors 2018, 18, 4011; DOI:10.3390/s18114011);
WO2017025921A1: “Aptamer Biosensors useful for detecting hormones, hormone mimics, and metabolites thereof” (Kumar, published 16.2.17).

### SUMMARY

According to a first aspect of the present invention, there is provided a saliva test system for performing an ELISA or ELONA test, a saliva receiver having an inlet channel for collecting from a user saliva comprising analyte, the saliva receiver to guide the collected saliva to an incubation chamber; the incubation chamber having bioreceptors for binding to the analyte and reagent, and for providing an incubated solution; the incubation chamber to incubate a substrate, the incubation to allow the substrate to react with a said bound reagent and thereby provide the incubated solution; a test chamber arranged to receive a said incubated solution from the incubation chamber, the test chamber having biosensing test electrodes to perform on the incubated solution a biosensing test; a controller to control the biosensing test electrodes to perform the biosensing test; and a user interface to indicate a status of a user based on a result of the biosensing test indicating presence or concentration of the analyte in the collected saliva, wherein the system comprises: a narrowing, such as a constriction, of the inlet channel, the narrowing for limiting a volume of the collected saliva; and a vent hole to assist flow of the collected saliva through the saliva receiver toward said incubation chamber, the vent hole coupled downstream of the narrowing and/or the incubation chamber and openable to enable flow of the collected saliva through the narrowing toward the incubation chamber.

The system may additionally or alternatively be suitable for tests other than ELISA or ELONA (Enzyme-Linked Oligonucleotide Assay). Regardless, the analyte may be a biomarker. Alternatively, the analyte may be a drug of abuse.

A preferred system may perform an assay comprising preferably all of the following steps: collecting a saliva sample in the inlet channel, incubating—separately or combined—the saliva sample and the reagent(s) (generally comprising conjugates) to allow competitive binding to the bioreceptors in the incubation chamber, incubating the substrate in the incubation chamber to provide an incubated solution to the test chamber, applying a test voltage to the biosensing test electrodes, and indicating a biosensing test result, e.g., by means of a visual and/or audible user interface (UI).

The incubation chamber may merely hold solution(s). However, control of conditions such as temperature, time and/or movement (e.g., shaking or rotating) may be involved in one or more incubation disclosed herein. The substrate may be introduced into the incubation chamber after the collected saliva (and possibly reagent(s)). The incubated substrate solution may then be passed to the test chamber and tested. In an ELISA/ELONA implementation, the substrate may be a substrate solution such as Tetramethylbenzidine (TMB).

Advantageously, using a vent hole to control the flow through a narrowing may improve, e.g., test convenience, speed, accuracy and/or reliability. By ensuring that an optimum amount of saliva is automatically collected, the collection may be performed efficiently and/or in a short amount of time, e.g., to reduce inconvenience to the user. The collection of an optimum amount of saliva may ensure an optimum concentration of reagent when the reagent is combined with the saliva.

The narrowing and/or vent hole may slow or prevent uncontrolled diffusion of (e.g., excess) saliva and/or diffusion of contaminant (e.g., saliva remaining from earlier tests) through the system, e.g., to the incubation and/or test chambers. For example, diffusion to the incubation chamber from the inlet channel may be reduced (e.g., substantially prevented). The narrowing may have at least one of: a length of about 1 mm to about 30 mm; a width of about 0.05 mm to about 1 mm; and a depth of about 0.1 mm to about 0.7 mm. As an example, and using the equation L_{D}≈\sqrt{DT}, where L_{D} is the distance travelled by a molecule (also known as the Diffusion Length) with diffusion coefficient D over a time τ; a protein with a diffusion coefficient of 10⁻⁶ cm²/s may take approximately 69 hours to diffuse over a length of 5 mm. This may be far greater than the order of time of an assay which may range from about 1 minute to about 30 minutes. Generally, it is merely desirable for the length of the narrowing to be at least sufficient to prevent molecules from diffusing across the distance at least within the time of the assay. A closed state of any other vent hole(s) mentioned herein, even if without an adjacent narrowing, may similarly reduce diffusion to the incubation and/or test chambers from other parts of the system, e.g., from any capillary pumps.

The reagent generally has conjugates each comprising a conjugated portion comprising the analyte, and another conjugated portion. Generally, a conjugate may be a protein comprising an enzyme portion and a hormone portion. The analyte may comprise a hormone such as cortisol, testosterone, progesterone or estradiol. The another conjugated portion may comprise an enzyme such as horseradish peroxidase enzyme (HRP). The substrate may comprise TMB. The incubation chamber may further receive a wash buffer to reduce reaction of the substrate and the reagent.

Fluid trapped in a channel of an open vent hole, such as the above vent hole preferably coupled by a channel adjacent the narrowing, is a potential source of error. Such fluid may mix by diffusion with subsequent solutions, effectively increasing the background to a saliva test. Flow into an open vent hole channel from a connected flow channel (e.g., the inlet channel) may be reduced by ensuring that a capillary pressure in the vent hole channel is lower than in the flow channel, e.g., by making the cross-sectional area of the open vent hole channel greater than that of the flow channel, by making surface(s) of the flow channel more hydrophobic and/or making the vent hole channel more hydrophilic.

The system may have at least one additional vent hole and microfluidic channel, said additional vent hole coupled by said microfluidic channel to at least one of the incubation chamber and said test chamber. At least one seal may be movable to open and/or seal any vent hole(s) mentioned herein, to thereby reduce or inhibit a flow, the flow preferably being of the collected saliva, a reagent solution comprising the reagent, the wash buffer and/or the substrate solution. Opening one or more such vent holes in sequence, e.g., by breaking one or more such seals, may control flow through the system, e.g., through the saliva receiver. Such control may effectively implement passive pumping, preferably without the need for further, active pumping mechanisms. Sequential opening of vent holes may assist multi step assays by controlling flow of a saliva sample through the system, for example to control flow into the incubation and/or test chambers.

In embodiments, a capillary pump may be coupled to pump the incubated solution toward the test chamber. Such an embodiment may utilise a microfluidic channel coupled to guide incubated saliva toward the capillary pump.

Preferably, at least one of the saliva receiver and the incubation chamber is for combining, e.g., mixing, of the collected saliva with the reagent to form a solution for the binding of the analyte and reagent to the bioreceptors in the incubation chamber.

The reagent may comprise a dry reagent disposed on a surface of said incubation chamber. Additionally or alternatively, the reagent may comprise dry reagent disposed on a surface of the saliva receiver to combine with the collected saliva, the surface preferably a surface of a channel such as the inlet channel. Thus, the system may comprise the reagent(s), e.g., on surface(s) of the saliva receiver (e.g., of a channel such as the inlet channel) and/or of a said incubation chamber, and either or both of such a channel or incubation chamber may be used for combining the saliva and reagent(s). Regardless, the reagent may comprise a dry reagent for forming a reagent solution when combined with the collected saliva.

There may further be provided a cap attachable to the saliva receiver to reduce (e.g., prevent) evaporation of the collected saliva, the cap preferably having a vent hole to reduce any increase of pressure in the cap and/or saliva receiver when the cap is being attached to seal an opening of the inlet channel. For example, during to the time required for such saliva and dry reagent to combine/mix, less of the saliva sample may then evaporate. The sealing attachment may involve inserting the saliva receiver at least partially into the cap (e.g., involving a press fit). Without a vent hole, an increase in pressure may cause the solution to flow through the system, e.g., through saliva receiver, at an undesired rate and/or direction, and/or may prevent a sufficiently tight fit of the cap such that a significant level of evaporation may continue.

There may further be provided at least one cartridge to contain at least one substance, the cartridge attachable to deliver the substance(s) to the saliva receiver, wherein the substance(s) comprise the saliva, the reagent(s), wash buffer(s) and/or substrate(s). (N.b., any ‘cap’ described herein that is used to collect or hold substances(s), e.g., saliva and/or reagent solution(s), may alternatively be referred to as a ‘cartridge’, however these may use a different design to any caps that are not provided to hold solution). Such reagent, buffer and/or substrate are preferably provided from the cartridge as solution(s), however the reagent(s) may additionally or alternatively comprise dry reagent. Multiple cartridges may be used to provide the substance(s), preferably respectively and/or sequentially (e.g., applied in turn to the saliva receiver, each to apply one or more such substance).

The cartridge may have an inlet for receiving saliva from a user, the cartridge attachable to deliver the received saliva to the inlet channel of the saliva receiver. In some embodiments, the saliva receiver may comprise at least one said cartridge. The inlet of the cartridge may comprise the inlet channel having the narrowing.

A said cartridge may have a vent hole to reduce any increase of pressure in the cartridge when the cartridge is being coupled to deliver a said substance to the saliva receiver. The vent hole may provide advantages as discussed above for a vent hole in a cap, for example when the coupling is achieved by inserting the saliva receiver at least partially into the cartridge, e.g., involving a press fit.

Cartridge(s) may have an external aperture to deliver the substance (e.g., saliva if collectable by the cartridge as indicated above) to a part of the saliva receiver, e.g., the inlet channel. A cartridge may have a hydrophilic inner surface preferably opposite the aperture. Such a surface may reduce loss of reagent solution and/or saliva during the attachment, e.g., during any insertion of at least part of the saliva receiver into the cartridge. The hydrophilic surface may ensure that an aperture of the saliva receiver to receive reagent or saliva from the cartridge is directly aligned with the reagent/saliva as it passes out of the cartridge aperture.

Wherein a substance to be delivered from a cartridge is a solution, the cartridge may comprise a hydrophobic surface to reduce spread of the solution from the hydrophilic inner surface, said hydrophobic surface comprising an internal and/or external surface of the cartridge, e.g., a surface coating.

There may further be provided the system having a separable component comprising a said cap and/or a said cartridge, the saliva receiver comprising a first detection electrode and the separable component comprising a second detection electrode located to contact the first detection electrode to enable detection of coupling of the saliva receiver to the separable component. Such detection of coupling, e.g., insertion of at least part of the saliva receiver into a cap and/or cartridge, may be used to indicate (e.g., by audible or visible alert) the proper coupling of the cap or cartridge, and/or to trigger the automatic opening of vent hole(s), e.g., at a (preferably respective) set amount(s) of time after the detection. Such automatic opening may reduce the time for which reagent and/or saliva delivered from a cartridge is held in contact with any reagent and/or saliva already present in the inlet channel of the saliva receiver, preferably reducing the diffusion between the two.

It is further noted that dried reagent(s) may be coated onto the inlet channel/chamber of a test strip. When the saliva is collected it may then mix with the dried reagent(s). A cap/cartridge may be attached to reduce evaporation of the saliva, thus potentially maintain an optimum saliva:reagent ratio for the biosensing test. The cap may be removed preferably after a predetermined desired time period. A cartridge, preferably attached in place of the removed cap, may contain a wash buffer to act as a buffer between the saliva and the substrate solution, to hinder the substrate from reacting with reagent(s). (The cartridge may also prevent the evaporation of fluids). Regardless, the reagents (specifically, conjugates) and saliva analytes may undergo a competitive reaction process for binding with the bioreceptors, e.g., antibodies in the incubation chamber. A preferably additional cartridge may be attached to introduce the substrate solution. Thus, a cap may be used between saliva collection and connection of a cartridge, primarily to prevent/reduce evaporation of the sample while it combines/mixes with the preferably dried reagents. Cartridge(s) may be later used to introduce additional substances, e.g., reagent(s), wash buffer(s) and/or substrate solution(s), into the test strip.

There may further be provided the system, wherein the bioreceptors comprise antibodies, the antibodies preferably for binding to a said analyte comprising a hormone such as cortisol, testosterone, progesterone or estradiol or a drug of abuse. Alternatively or additionally, the bioreceptors may comprise aptamers, the aptamers preferably for binding to a said analyte comprising a hormone such as cortisol, testosterone, progesterone or estradiol or a drug of abuse. Aptamers may be preferred as the bioreceptors for an ELONA test. As an example, the bioreceptor may comprise an estradiol-binding aptamer with a sequence 5′-ATA CGA GCT TGT TCA ATA CGA AGG GAT GCC GTT TGG GCC CAA GTT CGG CAT AGT GTG GTG ATA GTA AGA GCA ATC-3′ (SEQ ID NO: 1), or truncated from such a sequence.

The biosensing test(s) may comprise a test to, at least indirectly, detect or determine concentration in the collected saliva of at least one of: cortisol; testosterone; aldosterone; progesterone; estradiol; alpha-amylase; CRP; DHEA; and/or sIgA. Alternatively, the biosensing test(s) may comprise a test to, at least indirectly, detect or determine concentration in the collected saliva of a drug of abuse, such as alcohol, opioids, steroids, amphetamines, cannabinoids, benzodiazepines, NSAIDS, barbiturates, tricyclics, and ephedrines. In particular, the drug of abuse may be selected from one of cocaine, benzoylecgonine, cocaethylene, norcocaine, PCP, amphetamine, methamphetamine, cannabinoids, THC, carboxy-THC, heroin, codeine, morphine, 6-monoacetylmorphine (MAM), oxycodone, 3,4-methylenedioxyamphetamine (MDA); and 3,4-methylenedioxymethamphetamine (MDMA) or metabolites thereof.

Biosensing test(s) preferably comprise: an electrochemical impedance spectroscopy (EIS) test; a cyclic voltammetry test; and a chronoamperometry test; and/or a square wave voltammetry test. Any such test may use at least one working electrode, a reference electrode and a counter electrode. A working electrode is generally an electrode at which a cell reaction for the biosensing test takes place. The reference electrode may establish the electrical potential against which other potentials, e.g., that of a working electrode, may be determined. The counter and working electrodes together generally provide an electrical circuit in which current is measured (or applied), for example for measurement of current in response to an applied voltage for a biosensing test. Thus, a working/reference/counter electrode arrangement may provide a multiple electrode electrochemical cell for reactions expected to result in electric current flow, noting however that a two-electrode system may be used if the reference and working electrodes are combined. A cyclic voltammetry test may be carried out by cycling the potential of a working electrode, and measuring the resulting current. For the square wave voltammetry test, the current at a working electrode may be measured while the potential between the working electrode and a reference electrode is swept preferably linearly in time. The potential waveform may be viewed as a superposition of a regular square wave onto an underlying staircase. For the chronoamperometry test, the current at a working electrode may be measured while the potential between the working electrode and a reference electrode is stepped and the resulting current from faradaic processes occurring at the working electrode (caused by the potential step) is monitored as a function of time.

The saliva test system may perform the biosensing test(s) using dynamic measurement, wherein the biosensing electrodes comprise at least one working electrode and counter electrode, and the dynamic measurement comprises: measuring redox currents through a said working electrode at each of a first time and a later, second time. For each said working electrode the redox currents at each said time are preferably measured by voltammetry at respective, distinct frequencies of excitation of electric field between the working and counter electrodes. An analyte concentration of the saliva may be determined dependent on a ratio of the redox currents measured at the distinct frequencies at the first time relative to a ratio of the redox currents measured at the distinct frequencies at the second time.

According to another aspect, the system may be used to perform a method of performing an assay. Such a method may comprise controlling flow of the collected saliva through (at least part of) the system by opening vent holes in sequence, a said opening preferably comprising breaking or removing a seal. Sequential opening (e.g., piercing, removing or otherwise breaking seals) of the vent holes may occur automatically in response to the above-described detection of the coupling of the saliva receiver to a separable component (e.g., cap and/or cartridge) by means of the detection electrodes. Optionally, any previously opened vents may be resealed shortly before or shortly after opening the next vent hole in the sequence. Any opening and/or resealing may be performed manually by a user or may occur automatically, for example in response to detection of the coupling of cap(s) and/or cartridge(s). Such coupling may be of the saliva receiver to the cap after collecting the saliva sample, and/or of the saliva receiver to at least one cartridge containing substance(s) such as reagent(s).

Preferred embodiments are defined by the dependent claims.

### BRIEF DESCRIPTION OF THE DRAWINGS

For a better understanding of the invention and to show how the same may be carried into effect, reference will now be made, by way of example, to the accompanying drawings, in which:

FIG. 1 shows example elements of a saliva test system, combined in the illustrated embodiment to form a preferably integrated consumer device, i.e., test strip;

FIGS. 2a and b) show example microfluidics architectures of test strips;

FIG. 3 shows layout of a further embodiment of a microfluidic architecture of a test strip, including a cartridge with an inlet channel for collection of saliva;

FIG. 4 shows an example layout of electrodes that may be suitable for an embodiment, including biosensing electrodes and flow detection electrodes;

FIG. 5 shows an example process for conducting an, e.g., competitive ELISA, assay using a test strip embodiment such as that of FIG. 2a and/or 2b), the process comprising any one or more of steps a)-f);

FIGS. 6a) and b) together shows steps, any one or more of which may be carried out by a user during a process such as that of FIG. 5.

FIG. 7 shows steps, any one or more of which may be carried out by a user during an, e.g., competitive ELISA, assay preferably using test strip 300.

FIG. 8 shows a general computing structure, which may form or be comprised in any part of an embodiment system, e.g., test strip, mobile and/or remote computing device, may be implemented.

FIG. 9 illustrates an example ELISA assay comprising steps a)-c).

### DETAILED DESCRIPTION OF EMBODIMENTS

In an example test based on ELISA, bioreceptors may be attached to a surface. A sample comprising an analyte such as a hormone or a drug of abuse may be applied to the surface. An analyte-enzyme conjugate may further be present. A competitive reaction to bind the sample analyte and the analyte portion of the conjugate to bioreceptor binding sites may take place. A substance containing a substrate for the enzyme portion of the conjugate may further be present. The substrate may react with the enzyme portion of the bound conjugate, to result in a detectable signal usable for determining analyte concentration in the sample. An example ELONA test may be similar, however the bioreceptor may comprise an aptamer specific for detecting the target analyte. An embodiment implementing biomarker detection and/or measurement, for example based on ELISA or ELONA such as described above, generally uses conjugates that are synthesized externally and then used as reagents in the assay. A competitive ELISA assay of an embodiment may be based on competition between a target molecule (e.g., estradiol) and a conjugate molecule (e.g. HRP-estradiol) for binding to antibodies. The enzyme, e.g., HRP, component of the conjugate may be used as a label for signal generation in the presence of a substrate (e.g., TMB). Preferably, the conjugate is mixed with the saliva sample in a controlled way.

An example saliva test system comprises a saliva receiver to receive saliva from a user and guide the saliva to at least one incubation chamber. The saliva receiver may comprise a microfluidic system including an inlet channel with a narrower section before the saliva reaches incubation chamber(s). An open (or initially closed) vent hole may be located next to the inlet channel's narrower section. Additionally, a microfluidic channel may be present for saliva to flow through after incubation in incubation chamber(s), and a capillary pump provided at the end of the microfluidic channel. The system further comprises test chamber(s). A cap may prevent or reduce evaporation during saliva incubation and/or mixing in the incubation chamber or elsewhere in the system (e.g., elsewhere in a test strip). Cartridge(s) containing reagents solution(s) may be provided for coupling with the saliva receiver in such a way that reagents can flow through the inlet channel. Generally, the incubation chamber(s) are for holding the solutions, e.g., guided saliva, reagent, wash buffer and/or substrate, and the test chamber(s) receives an incubated solution from the incubation chamber via a microfluidic channel. Biosensing test electrodes for performing biosensing test(s) are provided in each test chamber. Generally, a biosensing test is for detecting or measuring concentration of analyte, which may be a biomarker or a drug of abuse, in the saliva. The incubation chamber(s) generally comprise at least one form of bioreceptor. A preferably integrated controller, e.g., microprocessor, at least controls the biosensing test electrodes to perform the biosensing test(s). A UI may indicate a status of the user, e.g., a hormone level, or a human condition or performance level such as stress status or level, based on the biosensing test(s).

Some preferred embodiments may involve a saliva sample being mixed with conjugate, in a separate container (e.g., in a cartridge) before being transferred into the test strip. Alternatively, the saliva sample may not be mixed with conjugate. Rather, the sample, and then the reagent, e.g., conjugate solution, may be incubated in the incubation chamber. The assay in this case may be based on “back filling” of the conjugate that will bind to the antibodies left available after incubation with the sample. A washing buffer may also be introduced between sample incubation and conjugate incubation. The biosensing, e.g., electrochemical test, may be carried out on a substrate solution after incubation of that solution in the incubation chamber, whereas saliva may be initially incubated in the incubation chamber and then moved away e.g., towards a capillary pump.

It is further noted that an embodiment may use dry reagent(s) (preferably provided within a test strip). Optional wash buffer(s), and then the substrate solution, may then be introduced.

A cartridge preferably containing reagent(s) may include an inlet channel to be used to collect saliva. Thus, a cartridge may be used as at least part of the saliva receiver.

Advantageously, electrochemical measurements can provide highly sensitive biosensing tests. Consequently, an embodiment may perform an assay with limited, e.g., small, sample volume. In turn, this may provide the advantage of limited time required for saliva sample collection. High sensitivity, preferably quantitative, testing for hormone(s) in saliva may thus be achieved by electrochemical measurements of the products of an assay in a preferably integrated test strip. A preferred test strip with an integrated fluidic system, preferably in combination with cartridges, may carry out a competitive assay on saliva using an electrochemical sensor to detect the products of the assay with low, preferably sub-picomolar, limit of detection. This may be particularly advantageous for hormone salivary tests for human performance optimisation, e.g., based on detecting estradiol, cortisol, testosterone and/or progesterone.

An embodiment may obtain a low limit of detection for hormones in a saliva matrix by means of an electrochemical sensor, for example when an ELISA or ELONA test is carried out on the saliva sample within a test strip. In this regard, the inventors have observed sub-picomolar limit of detection when using electrochemical measurement methods (such as square wave voltammetry method) to measure the concentration of HRP enzyme in the presence of its substrate TMB. Since the HRP-TMB reaction may generally be the amplification system at the basis of ELISA and/or ELONA assays, such observation may open the opportunity for highly sensitive electrochemical biosensing. Furthermore, the inventors have devised a test strip architecture where the elements of the assay can be integrated in such a way that the time for saliva collection and the number of actions required to the operator are minimised.

Cartridges may be used to add a sequence of substances into the saliva testing system according to a specific assay protocol. In the case of a competitive ELISA assay for estradiol, such substances may comprise, e.g., estradiol-HRP conjugates, washing buffers and/or a TMB substrate.

The saliva test system may be provided in the form of a microfluidics system within a test strip. At least part of a saliva receiver of the system may be a separate element initially not attached to the test strip. Regardless, the saliva receiver may be used to collect the saliva sample from a user's mouth, e.g. through a capillary tube. After the sample's collection, the receiver part (if separate) may then be connected by the user to the test strip. The saliva receiver may hold reagents that may be mixed with the saliva sample after the sample has reached a chamber of the saliva receiver via the inlet channel. Any reagent(s) may be either in dry form (preferably dried onto a surface of the saliva receiver, e.g., of a channel or chamber thereof) or in solution (e.g. in a buffer solution). If the reagent(s) are in solution, the solution may be provided in a separate chamber (e.g., reservoir) within the saliva receiver, connected with the aforementioned channel or chamber of the saliva receiver via one or more microfluidic channels, or otherwise provided from a cartridge into which the test strip may be inserted. Alternative or additional reagent(s) may be collected from a cartridge. For example, reagent in the saliva receiver may comprise HRP conjugates such as HRP-estradiol conjugate, which may compete with a target analyte in binding to the bioreceptors, and/or HRP-streptavidin conjugate that may bind to a biotinylated probe.

The test strip may include chambers and respective vent holes, at least one of which may initially be closed. For example, a first chamber may be an incubation chamber where the saliva sample—which may be previously mixed with reagent—is incubated with bioreceptors such as antibodies and/or aptamers. A second chamber may be a test, or sensing, chamber where an electrochemical measurement is carried out on a product of the assay. Bioreceptors may be immobilised onto a surface within the incubation chamber. Reagent(s) such as TMB substrate may be dried onto a surface of the test chamber. A first open vent hole may allow flow of the saliva sample into the first chamber. A second vent hole may be initially closed and then opened when the incubation in the first chamber is finished and the solution is to flow into the second chamber. In this regard, any open vent hole—generally closed initially to reduce/prevent onwards sample flow through the system—may be subsequently closable, e.g., by means of a manually and/or automatically placed seal.

In an example embodiment, the saliva receiver may include reagent(s) to condition the saliva sample. For example, reagent(s) may be provided to reduce or inhibit the activity of salivary enzymes (such as lactoperoxidase) in order to reduce or prevent interference in the assay due to interaction, e.g., with TMB. For example, thiocyanate salts (e.g. sodium thiocyanate) may be dried within the saliva receiver. If mixed with the saliva sample, salivary lactoperoxidase molecules may be saturated by thiocyanate and therefore may be inactive.

Glucose oxidase conjugates (such as glucose oxidase-estradiol conjugate) may be used in a competition assay and the reaction with glucose may be used for electrochemical detection. Advantageously, glucose is generally not affected by the presence of salivary enzymes, so that interference effects may be avoided.

The bioreceptors may comprise antibodies such as estradiol binding monoclonal antibodies. The antibodies may be immobilised on a plastic surface within the test system, e.g., on PMMA or polystyrene or polycarbonate, or on a gold surface such as a thin film gold on plastic.

Similarly, the bioreceptors may comprise a hormone-binding aptamer modified with a thiol group or with an amine group at the 3′ end. The aptamer may be hybridised with a complementary strand, and the complementary strand may be labelled with biotin group or with HRP.

An example biosensing test may use a gold area within incubation chamber(s) functionalised with aptamers that may selectively bind to a specific analyte (e.g., estradiol) in a saliva sample. The aptamers may be hybridised with biotinylated complementary strands (the probes). The binding of the analyte to the bioreceptor may determine a displacement of the probes. A reagent such as streptavidin-HRP conjugate may be introduced and may bind to the biotin group of the probes, and a reagent such as TMB may be introduced to provide a substrate for the HRP enzyme. The system may use electrochemical measurements such as cyclic voltammetry, square wave voltammetry, chronoamperometry, etc. to measure the TMB solution after reaction with the HRP enzyme.

At least one biosensing test may comprise an EIS test, a cyclic voltammetry test, a SWV test, and/or a chronoamperometry test, and/or may be to detect or determine concentration of at least one biomarker such as: cortisol; testosterone; progesterone, aldosterone; estradiol; alpha-amylase; CRP; DHEA; and/or sIgA. Additional or alternative targets may be drugs of abuse, and/or viruses.

The biosensing test electrodes may comprise working electrode(s) and counter electrode(s), and a reference electrode may also be present. In one example, at least one working electrode is positioned on an inside surface of a test chamber. A counter electrode may be positioned at/on the test chamber and proximate the working electrode(s). Furthermore, a reference electrode may be positioned at/on an end of the test chamber.

More specifically, the counter electrode may be positioned between working electrodes, e.g., centrally between one or more pairs of working electrodes. The saliva test system may further comprise saliva detect electrodes as discussed herein.

The system may comprise an electrical interface to allow external control and/or measurement of voltage and/or current on the biosensing electrodes. The controller may set a voltage (or current) between electrodes, and the resulting current (or voltage) may be read by electronics (which may be internal or external to the system, e.g., to a test strip). The control and/or reading may be performed by a reader device having a physical interface for electrical connection to the biosensing electrodes, e.g., allowing an end of the test strip to be inserted into the reader.

Correspondingly, a reader device may have at least one electrical interface, for receiving saliva detect test signals and/or saliva biosensing test signals; and a controller to detect saliva based on at least one said saliva detect test signal and/or to, preferably when saliva is detected, determine a measure of biomarker concentration based on at least one said saliva biosensing test signal. An optional UI of the system may indicate a user status based on the determined measure. The reader may be configured to physically receive and electrically couple to at least an end of a saliva test strip such as that described above. The reader device may then control electrical signals on the interface to the test strip, to enable detection of the saliva and/or to perform biosensing test(s).

Consistent with the above, a saliva test system embodiment may comprise: a test strip having an electrical interface, the test strip comprising, e.g., the saliva receiver, incubation and test chamber(s), biosensing test electrodes and/or preferably saliva detect electrodes; and a reader apparatus having the controller and an electrical interface to couple to the electrical interface of the test strip. The reader apparatus may control and measure voltage and/or current on the saliva detect electrodes and/or biosensing test electrodes by means of the electrical interface to perform, respectively, a saliva presence test and/or biosensing test(s). The reader apparatus may thereby determine user status (e.g., hormone level, or a human condition or performance level such as stress level) based on the biosensing test(s), wherein the test strip and reader apparatus are separable. The reader apparatus may further comprise the UI to indicate user status. Thus, the reader and test strip may each be supplied alone, together as a saliva test kit, or integrated in a single device. In any of these cases the user may use the combination of reader and test strip as a handheld device. The test strip may—disregarding the electrodes—have substantially no electronics and/or may be disposable, so that a new test strip can be inserted into the reader device for each saliva sample.

Any embodiment of the saliva test system may be suitable for sampling and analysing, and indicating the analysis result, e.g., user status, at a point of care (POC). The POC is generally the location of the user when the saliva receiver is inserted into the user's mouth. For example, this may be at a user's home, at a sports facility, at a military base, etc. In other words, there is no need for the POC to be a specialist medical facility such as a hospital or laboratory. The saliva sample can generally be taken at any POC and the user status result provided after only a short delay. No trained medical profession may be required to be present.

Further advantageously, the system may be portable, preferably comprising a single handheld device albeit wherein the device may comprise separable parts such as a test strip and reader, and/or wherein UI(s) may additionally or alternatively be provided at a remote computing device such as laptop, cloud computing device and/or mobile phone.

Various different biomarkers in saliva may be detected by embodiments, and the biosensing test(s) may detect and/or measure the biomarker concentration. The biomarker detection may involve interaction of an analyte with a biological element. For example, detection of a biomarker comprising the hormone cortisol may allow application of an embodiment to detecting and/or measuring a human condition or performance level, e.g., stress of a user. The user status may then be indicated as, e.g., ‘stressed’, ‘not stressed’ or as a stress level. However, the system may be a more general salivary hormones test system and/or the status of the user may be any salivary biomarker level, desirably based on detection and/or measurement of estradiol, progesterone, testosterone and/or cortisol.

Alternatively, different drugs of abuse may be detected by embodiments, and the biosensing test(s) may detect and/or measure the concentration of the drug of abuse. As used herein, a drug of abuse may refer to alcohol, opioids, steroids, amphetamines, cannabinoids, benzodiazepines, NSAIDS, barbiturates, tricyclics, and ephedrines. In particular, the drug of abuse may be selected from one of cocaine, benzoylecgonine, cocaethylene, norcocaine, PCP, amphetamine, methamphetamine, cannabinoids, THC, carboxy-THC, heroin, codeine, morphine, 6-monoacetylmorphine (MAM), oxycodone, 3,4-methylenedioxyamphetamine (MDA); and 3,4-methylenedioxymethamphetamine (MDMA) or metabolites thereof. Accordingly, detection of the drug of abuse in a patient may be particularly useful to rapidly determine the presence and/or amount of a drug of abuse in a sample.

Any of the saliva detection and/or biosensing tests may be impedimetric and/or electrochemical test(s) and may involve applying a predetermined voltage signal between electrodes and measuring the resulting current signal in those electrodes.

The UI(s) for indicating user status indication(s) may be provided on a separable reader device as described above. However, the status may additionally or alternatively be indicated by a UI at a remote location, e.g., via a wireless mobile device and/or cloud networking component coupled to a reader device. The indication may be, e.g., on a display of a reader device connectable to a test strip, and/or by wireless communication (e.g., using BlueTooth™) to a remote computing device such as a mobile phone that displays the status using an app. In a preferred embodiment a reader device, whether connectable to or integrated with the test strip, does not have any display as such, and may just have LEDs to show basic messages like, e.g., ‘power on’ and/or ‘measurement ongoing’, etc. User status results may then be shown by a mobile app in a mobile device connected to the reader device. Thus, an example UI may comprise a set of LEDs, preferably on a separable reader, indicating a status of a saliva test process (comprising saliva detect and/or biosensing) such as power, connection, saliva present, measurement ongoing and/or measurement done etc. Additionally or alternatively, a UI may comprise a mobile app where quantitative results can be shown on a display. The mobile app may be run on a mobile device such as a mobile phone and may be in wireless communication with the reader.

An embodiment may comprise microfluidic saliva flow channel(s) to draw saliva in by means of capillary action. In the saliva receiver, such channel(s) may comprise the inlet channel and/or may guide saliva to the incubation chamber(s). Such saliva flow channel(s) may have a width greater than about (e.g., exactly) 1 mm and/or less than about 30 mm. The height of the channel may be, e.g., greater than about 100 micrometers and less than about 700 micrometers. The inlet channel may comprise a narrowing (e.g., a narrower end of the channel or a constriction of the channel) between an outermost end of the inlet channel end (which may be directly exposed to the environment) and incubation chamber(s). Such a narrowing or constriction may have a minimum width in the range of about (e.g., exactly) 50 um to about 1 mm. A (preferably closable) vent hole may be included and connected to the constriction in order to allow the saliva to flow through the inlet channel until the position of the vent hole. Additional channel(s) may connect the incubation chamber to capillary pump(s) and allow saliva and/or other solutions to flow away from the incubation chamber upon opening of vent hole(s) positioned after the capillary pump(s). Another channel may connect the incubation chamber to the test chamber, and another channel may connect the testing chamber to a capillary pump, in order to allow the flow of a solution between the two chambers upon opening of a vent hole located after such capillary pump. Such vent hole(s) may be sealed e.g. by tape and may be opened by the user e.g. by peeling off the tape and/or by piercing the tape.

At least one pair of the biosensing test electrodes may be configured to apply an electric field to the incubated solution in the test chamber.

Preferably, at least one internal surface of the saliva receiver and/or at least one said incubation chamber is hydrophilic. This may enhance flow of the saliva.

The system may include separate elements in the form of one or more caps or cartridges that may be used to reduce evaporation of the solution(s) present in the inlet channel and/or to load a sequence of different solutions into the test strip. Such a cap/cartridge may include a vent hole to avoid overpressure upon insertion of the strip in the cap. The cap's inner volume may be larger than the volume occupied by the part of test strip inserted in the cap, in order to minimise air pressure that could hamper the test strip insertion. For example, the cap's inner volume could be twice the volume of the inserted test strip's part. A cap/cartridge for holding liquid substances may comprise a hydrophilic inner surface at the side opposite to an aperture, whereas all the rest of the inner surface may be hydrophobic. This may allow a drop of the substance to be confined on the hydrophilic surface. For example, the hydrophilic surface may be made of a hydrophilic adhesive film, and the hydrophobic surface may be made of PTFE or PMMA. The outer surface of the saliva receiver may be hydrophobic; this may to reduce any uncontrolled spreading of liquids on the outer surface when the saliva receiver is collecting, e.g., saliva from the user's mouth and/or other solutions from the caps. For example the external surface of the test strip may be made hydrophobic by PTFE spray coating or dip coating.

The test strip may have multiple, preferably 3 or more, of the following layers: a first layer as a hydrophilic adhesive top layer; a second layer as a polymer layer for microfluidics; a third layer as a double-sided adhesive layer; and/or a fourth layer as a base layer comprising the electrodes, to together define cavities comprising the saliva receiver, at least one said incubation chamber, at least one said test chamber, and preferably one or more capillary pumps. The second layer may comprise engraved regions on either top or bottom side, and/or may comprise vias to allow a solution to flow from a top side channel to a down side channel. Vent hole(s) may enable air to vent to the outside environment to allow pressure equalisation and/or capillary action to assist the flow of saliva and/or other solutions. More specifically, the test strip may have a first layer (e.g., top layer) to favour solutions' flow on a hydrophilic surface, a second layer into which channels and chambers are carved on either side of the layer and a via is opened to connect channels and/or chambers on the top side to channels and/or chambers on the bottom side of the layer, a third layer (double-adhesive) and a fourth layer (e.g., base) to together define the cavities required for the saliva testing.

A preferred situation may comprise a single film double-side adhesive where preferably at least one adhesive is hydrophilic rather than a laminate of two different tapes. The test strip may then have 3 layers: a first layer as a polymer layer for microfluidics, a second layer in the middle as a laminate layer preferably comprising a hydrophilic layer and/or a double-side adhesive, a third layer as a base layer comprising the electrodes, to together define cavities comprising the saliva receiver, incubation chamber(s), test chamber(s), and preferably capillary pump(s).

A saliva test system may comprise at least one of: a microfluidic inlet channel to guide the saliva; a constriction of the inlet channel with an open vent hole connected to the constriction, to allow a controlled volume of the received saliva and at least one said reagent to combine; at least one vent hole to initiate flow of the mixed saliva to a said incubation chamber and/or flow of a washing buffer from an inserted cap though the inlet channel up to the inlet channel constriction upon opening of the vent hole. At least one vent hole may be used to initiate flow of a substrate solution from an inserted cap though the inlet channel into the incubation chamber. At least one vent hole may initiate flow of the substrate solution from the incubation chamber into the test chamber and/or biosensing test electrodes. Preferably, at least one said microfluidic inlet channel has a width of between about 1 and about 10 mm, and preferably a height of between about 100 micrometers and about 700 micrometers, at least one said inlet channel's constriction has a width of between about 0.05 mm and about 1 mm, and at least one said incubation chamber has a width of between about 0.1 mm and about 5 mm.

A saliva test system such as the above (preferably comprising a test strip) may allow: collection of saliva; saliva conditioning (e.g., using microfluidics, and dry reagent(s)) and/or wet reagent(s) in a liquid reservoir); and/or biosensing measurement. The test strip, which may be insertable into a corresponding reader, preferably actuates (e.g., in response to the insertion and/or by, e.g., a vibrator integrated within the reader) the mixing of saliva within the saliva conditioning system and initiates flow onto the biosensor, e.g., into test chamber(s) and/or onto biosensing test electrode(s). The opening of vent hole(s) to initiate respective flow(s) may be achieved manually (e.g. by peeling off tape) or may be automated (e.g., by piercing the top layer with needles preferably actuated through a separate reader device). A controller, internal or external to the test strip, may control the biosensing measurement. Furthermore, a UI (e.g., on a phone and/or personal computer) may display the sensing and/or diagnosis results and/or guide the user. Preferably, the test strip samples the saliva, stores it for a short period of time and then, e.g., upon insertion into the reader if a separable reader is provided, the (optional) conditioning and/or the measurement is initiated by the controller. To assist such automation, saliva detection electrodes (if present) could be activated in response to the test strip being inserted into a separable reader.

Preferably by means of a saliva test system as described herein, a method may be performed for testing saliva for the presence of at least one analyte, such as a biomarker or drug of abuse. Such a method may comprise: collecting and mixing saliva with at least one reagent; performing a competitive assay based on competitive binding to bioreceptors of the reagent against the target analyte, at least one biosensing test on the collected saliva based on electrochemical measurement of the assay's product to measure concentration of at least one analyte, such as a biomarker or drug of abuse in the saliva, the at least one biosensing test using at least one bioreceptor; determining a status of the user based on the at least one biosensing test. Any such bioreceptor may comprise a hormone-binding aptamer and/or antibody. The biosensing test may detect a hormone such as cortisol, testosterone, progesterone or estradiol. The at least one said reagent may comprise at least one of: enzyme conjugates such as HRP-estradiol and/or HRP-streptavidin and/or glucose-oxidase estradiol and/or glucose oxidase streptavidin.

Advantageously, embodiments may provide one or more of: real-time results; quantitative results; high sensitivity; portability; ease of use; testing directly at a training site or POC; no external sample pre-treatment; or human status/performance analysis without requiring blood sampling. In this regard, the composition of the dry or liquid reagent(s), e.g., buffer diluent, may be specific to a particular bioreceptor (e.g., aptamer) used for biosensing, to small molecule (e.g., steroid hormone) targets being measured, and/or to the sensing architecture being used. The reagents may comprise, e.g., enzyme-target conjugates for competition assay, salivary enzymes inhibitors to prevent interference, chelation agent for chelation of divalent metal ions; monovalent salts; buffer salts to adjust the pH; and/or target dilution strategy utilising antibodies.

Where an embodiment comprises a test strip to be inserted into a reader device, the strip may be regarded as disposable. Saliva may be directly sampled using the test strip, or alternatively sampled using a separate component and then dispensed onto the test strip. Analyte concentration may then be analysed and preferably communicated (e.g., wirelessly) from a test strip reader device to an output device such as a mobile device or other computer. A system may thus provide a convenient, portable and/or easy-to-use system to monitor levels of chosen biomarkers or the level of a drug of abuse in an individual's saliva.

The system may perform a number of test(s) without the requirement of a lab environment or trained professionals. Furthermore, no saliva sample preparation outside the apparatus may be required. No remote chemical or electronic processing, e.g., dilution, centrifugation, pipette sampling, and/or external analysis of the saliva, may be necessary. An on-the-spot test result may therefore be provided after only a short delay, e.g., 10 minutes or less. The saliva may be automatically collected from the user's mouth and analysed for concentration of specific molecules by means of electrochemical measurement(s) that are suitable for biosensing test(s) to detect the target biomarker(s) or to detect drugs of abuse. Active intervention of the user may be limited to inserting different caps onto the test strip at different times as requested, e.g., by a UI software such as a mobile application.

Therefore, the apparatus may be suited to point-of-care (POC) health and/or wellness diagnostics. For example, the system may aid testing human performance, e.g., a stress condition of an athlete during training and/or competitions, or for monitoring military personnel enduring harsh, dangerous and/or traumatic working conditions. The test result may depend only on physical saliva sampling and analysis, and consequently accuracy is generally higher than with other methods, such as questionnaires where factors such as any perceived stigma associated with physical and/or mental health issues may affect a subject's response.

In specific embodiments, an example saliva test system may have a reader that is integrated within or attachable to a test strip. Thus, an embodiment may comprise:
a test strip for collection of saliva from the mouth and preferably including an electrochemical transducer (comprising at least electrodes) for the analysis e.g., biosensing, of saliva. The strip may comprise a saliva conditioning system in the form of an integrated microfluidics system, for preparation of the saliva sample before a measurement is carried out through an electrochemical transducer generally comprising biosensing electrodes; and/or
a reader device into which the test strip is inserted and which controls the electrochemical transducer and/or other electrodes included in the test strip. The reader device may be wirelessly connected to a remote computing device such as a smartphone and may be configured to transmit data to such a device. The reader device is preferably designed to be portable. (The functionality of the test strip and reader device may however be provided within a single integrated unit without the test strip being separable from the reader electronics).

The remote computing device (e.g., mobile phone, laptop or desktop computer, or cloud computing component such as a network server) may receive and/or distribute test outputs from a reader device. The reader device and test strip, which may be integrated, may together form a preferably handheld saliva receiving device; the computing device preferably being wirelessly coupled thereto. The computing device and/or the reader device may have a first UI to indicate when saliva is detected and/or a second UI to indicate a user status based on biosensing test(s). Any such interface may be audio and/or visual, e.g., a display on the computing device and/or LEDs on the reader device.

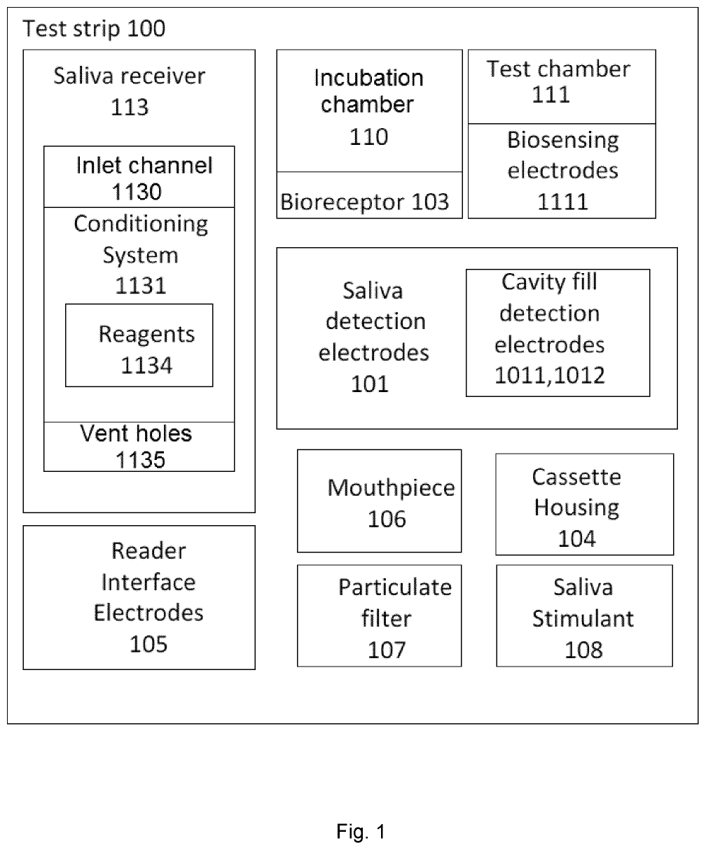

A system preferably in the form of a test strip 100 is shown in FIG. 1, wherein any one or more of the illustrated elements may be present. Such elements may include any one or more of: incubation chamber 110 with bioreceptor 103; test chamber 111 and associated biosensing electrodes 1111; electrical interface(s) 105 for example for connection to an interface of a reader apparatus; saliva receiver (e.g., mouthpiece and/or saliva inlet channel) 106 with an inlet channel 1130; the saliva receiver including a saliva conditioning system 1131 and reagents 1134; vent holes 1135 to control the flow of solutions within the test strip; caps to prevent saliva evaporation after collection and/or to load solutions into the test strip; caps detect electrodes 101, 1011 and/or 1012 to detect the presence of specific caps inserted onto the test strip and/or to trigger the opening of corresponding vent holes(s). The reagents 1134 may be, e.g., enzyme-target conjugates for competition assay, salivary enzymes inhibitors, chelating agents, salts and/or buffers, and may be held in the inlet chamber and/or in a reservoir connected to the inlet chamber. The incubation chamber 110 may optionally form part of the conditioning system 1131.

Similarly, test strip 100 may include electrodes for: saliva inlet flow detection and/or control; detection and/or control of saliva flow onto electrochemical sensing electrodes e.g., for biosensing; cap(s) insertion detection and/or control; electrochemical measurements such as voltammetry or chronoamperometry e.g., for the biosensing; and/or electrical interface with a reader device.

The test strip 100 may automatically extract saliva when inserted into the mouth. This may use a capillary action to assist flow of the saliva to an incubation chamber, for example by means of microfluidics. This may avoid the need to, e.g., provide a pump means for automatic extraction of the saliva.

A corresponding reader device may provide, control and/or read one or more of: electrochemical measurement(s), e.g., for the biosensing (square wave voltammetry, cyclic voltammetry, chronoamperometry); visual indicator(s) of sensor status; communication to a mobile device, a computer and/or to the cloud; and electrical interface(s) to a test strip 100.

A test strip may be suitable for integrated sampling and measurement of saliva directly from a person's mouth. A measurement flow process may be implemented to automatically determine the time when to start test(s), e.g., any electrochemical measurement(s) such as for sensing biomarker(s). It may be automatically determined whether the saliva has been correctly sampled and/or whether the test is or will be valid. The test process may be initiated for example by pressing a power button of a reader device, inserting the test strip 100 into the reader, and placing the test strip in the mouth. The test process may comprise insertion of caps to prevent saliva evaporation after collection and or to load solutions into the test strip. The test process may comprise opening a sequence of vent holes in order to allow the flow of saliva and/or other solutions through microfluidic channels and chambers within the test strip.

The test process may comprise cap detection test(s) to check the presence of different cap(s) and/or to trigger the opening of different vent holes. For example, the or each cap may comprise electrodes interconnected by means of a resistor. The electrodes may come into contact with cap detection electrodes upon mounting/insertion on/into the test strip. An impedimetric test may be carried out and the specific resistance associated with a specific cap may be measured. The result of the measurement may be used to recognise the proper insertion of the cap and/or to trigger the opening of a vent hole, e.g. by instantaneously operating an actuator pushing down a needle at a specific position to pierce a specific vent hole, or by melting an electrically conductive area covering a vent hole because of the current flow. Such a feature may minimise the time for which a new solution within a cap is kept in contact with any liquid already present in the inlet chamber of the test strip, thereby minimising the diffusion between the two liquids.

The process of collecting saliva and/or other solutions into the test strip by opening a sequence of vent holes to carry out a competitive assay such as ELISA/ELONA may have any one or more of the following steps:
saliva fills up the inlet chamber to open vent hole 1 connected to a constriction and is mixed with reagents (such as enzyme conjugate);
cap 1 may be inserted to avoid evaporation;
cap 1 may be removed and a cap 2 carrying washing buffer may be inserted and vent hole 2 preferably opened to allow saliva to fill the incubation chamber;
cap 2 may be removed and a cap 3 carrying substrate solution (such as TMB) may be inserted, and preferably a vent hole 3 is opened to allow washing buffer to flow through the incubation chamber and substrate solution to fill the incubation chamber;
vent hole 4 may be opened to allow the substrate solution to flow from the incubation chamber into the test chamber.

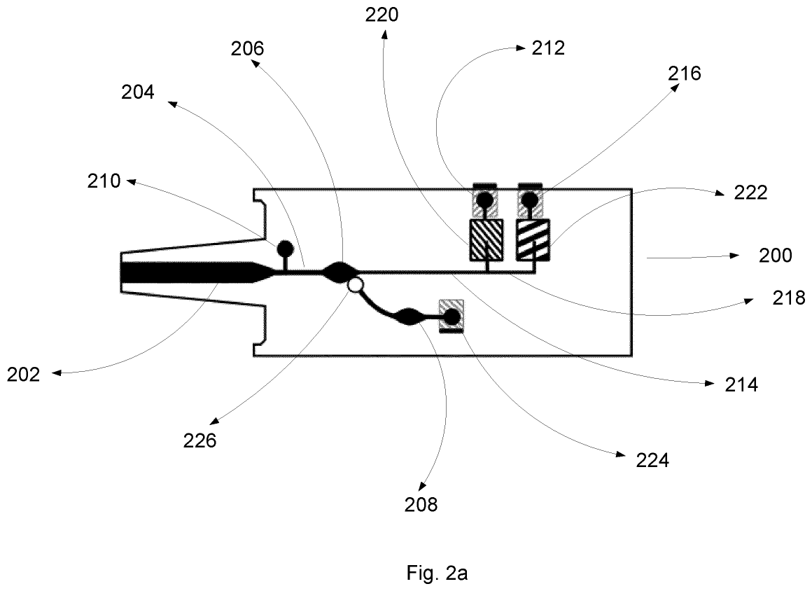

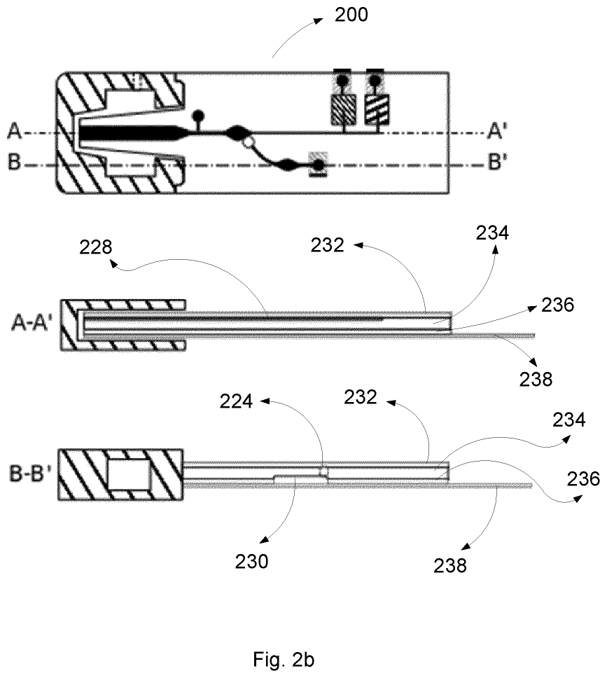

FIG. 2a shows a top view of an example test strip 200, in which a competitive ELISA process may be performed. The test strip 200 comprises an inlet channel 202 with a constricted section 204. The constricted channel 204 connects to an incubation chamber 206. The test strip further comprises a first vent hole 210 that is initially open and is connected to the constricted channel 204. A first microfluidic channel 218 extends between the incubation chamber 206 and a second vent 212 that is initially closed, while a second microfluidic channel extends between the second vent 212 and a third vent 216, with the third vent 216 also being initially closed. In this embodiment, the test strip 200 is further provided with a first capillary pump 220 and a second capillary pump 222 that connect the second and third vents 212, 216 to the first and second microfluidic channels 214, 218 respectively. The capillary pumps 220, 222 may improve the flow rate of the saliva sample. As for other specific features such as the vias, one or more of the vents, etc. the capillary pump(s) are optional. The flow of the fluid may be provided solely by capillary action through the constriction 204 and/or microfluidic channels 214, 218, and the flow may be controlled by opening the vent holes in sequence.

The incubation chamber may further be connected to a test or sensing chamber 208 by means of a via 226 from the top engraved channel to the bottom engraved channel (see FIG. 2b). The test chamber 208 is itself connected to a fourth vent hole 224 that may be initially closed. Vents that are initially closed may be sealed with tape or any other air tight means. Opening the vents may consist of removing or puncturing the air tight seal. This process may be manual (i.e. performed by the user) or automatic.

In order for effective passive pumping due to capillary action to occur, air trapped within the microfluidic channels generally has an evacuation route. Otherwise, the pressure from the trapped air may prevent the fluid from flowing along the channel. The various vents in the test strip provide such an evacuation route. Due to this, the sample in the test strip may only flow through the constriction 204, via 226 and/or microfluidic channels 214, 218 when a vent hole downstream from the sample's current position has been opened.

As an example, a saliva sample introduced to the test strip 200 may only flow as far as the open vent hole 210, and may not flow into the incubation chamber or the microfluidic channel 214 until the vent hole 212 is opened. Equally, the sample may not flow through the via 226 or the microfluidic channel 218 until the corresponding downstream vent holes 224, 216 have been opened.

Depending on the assay being performed, fewer or additional vent holes may be required, as in general there is a positive correlation between the number of vent holes and the number of steps that may be performed in an assay using the test strip. Further referring to FIG. 2a, a preferred order of operation, wherein any step(s) may be omitted, is: 1) add saliva; 2) open vent 212; 3) add conjugate solution; 4) open vent 216; 5) add wash buffer; 6) add (e.g., TMB) substrate solution; 7) open vent 224 to allow substrate, e.g., TMB, that has incubated in chamber 206 to flow into chamber 208 for measurement with the test electrodes.

FIG. 2b shows a detailed cross section of the top engraved channel 228 and the bottom engraved channel 230. The test strip comprises of several layers, including a hydrophilic film 232, a microfluidic substrate 234, a double-side adhesive 236 and an electrode film 238.

The substrate 234 may be structured to form one or more cavities between the substrate and the superstrate which may become at least partially filled by the sample under analysis. Such cavities may comprise an inlet channel 202, components of a microfluidic conditioning system, incubation chamber(s) 206 and/or test chamber(s) 208. The first layer may comprise a vent hole to allow the saliva to flow. The surface chemistry of the substrate and/or the superstrate may be hydrophilic to promote the flow of the sample fluid to fill the cavity volume(s) by capillary action. The substrate may be structured to form one or more cavities between the substrate and the base layer which may become at least partially filled by one solution coming from the incubation chamber through a via.

The hydrophilic film 232 may be a polyester film with pressure-sensitive adhesive with water contact angle of less than about 5 degrees and a thickness of about 0.05 mm to about 0.2 mm. The microfluidic substrate 234 may be constructed out of materials including (but not limited to) polymethylmethacrylate (PMMA), polypropylene (PP), cyclic olefin copolymer (COC) and/or polytetrafluoroethylene (PTFE) and may have a thickness of about 1 mm to about 3 mm. The depth of the, e.g., carved, cavities within the substrate 234 may be between 200 and 700 micrometers. The width of an inlet chamber (e.g., comprising saliva receiver and/or test chamber) may be between 1 and 10 millimeters, with a constriction with a width between 0.05 and 1 mm. The superstrate 234 may have an adhesive layer(s), or there may be a separate adhesive layer 236 between the superstrate and the substrate. This layer 234 may be fabricated via various methods, such as injection moulding, hot embossing and/or laser cutting/ablation, among others, and will generally have a thickness of about 1 mm to about 3 mm. The double-side adhesive 236 may be a polyester film with a pressure-sensitive adhesive, and a total thickness of about 50 to about 150 mm. The electrode film 238 may be a polyester film with sputtered metal film (e.g. gold, silver) with a sheet resistance of between approximately 1-20 Ω/□.

An additional hydrophilic film may be included between the microfluidic substrate 234 and the double-sided adhesive 236 in some implementations to further promote flow into the bottom-engraved channel 230. Additionally, one or more labels may be included on the top surface to identify the device, and/or to identify the vent holes to aid the user.

Additionally or alternatively, embodiments may comprise a PMMA layer with fluidics on the both top and bottom sides, a via to connect the top and bottom sides, and/or a layer with hydrophilic surface. Additionally or alternatively, embodiments may comprise a laminate middle layer between a PMMA top layer and a polyester base layer with sputtered metal films, wherein the laminate may include a hydrophilic adhesive film and/or a double-side adhesive film. In embodiments, the substrate layer may comprise through holes and channels providing walls for microfluidics, wherein the top layer and base layer may provide the top and bottom surface for the cavities.

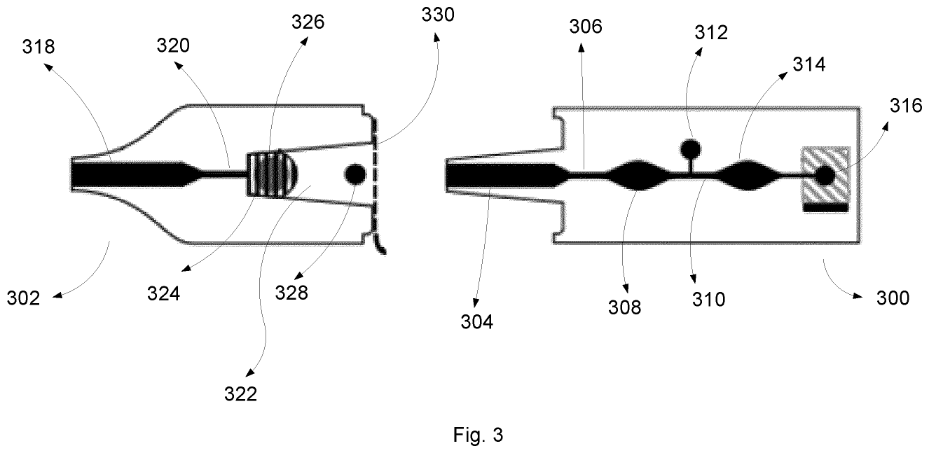

FIG. 3 shows a further example test strip 300 for performing a competitive ELISA process. In this embodiment, a detachable saliva collection cartridge 302 is used to collect the saliva sample. The test strip 300 may include an inlet 304 connected to a first microfluidic channel 306, an incubation chamber 308, a test chamber 314 connected to the incubation chamber 308 via a second microfluidic channel 310, and/or an open vent 312 connected to the microfluidic channel 310. An additional closed vent 316 may be connected to the test chamber 314, and may be sealed with tape or other air tight means. The closed vent 316 may be opened by piercing/removing the tape, or otherwise breaking the air tight seal.

The cartridge 302 may be used for collecting a sample of saliva, and may include an inlet channel 318 with a constricted section 320, and preferably a saliva collection chamber 322. The saliva collection chamber 322 may include a hydrophilic region 324 and preferably contains dried reagents 326 and/or a vent hole 328. The chamber 322 may be sealed with tape or other air tight means 330, the tape or other air tight means 330 preferably forming the surface of the chamber opposite the inlet aperture. Saliva collected in the cap 300 may mix with the dried reagents 326 in the chamber 322. When inserting the test strip 300 into the cap 302, the tape or air tight means may be pierced or otherwise broken, allowing the saliva sample to flow into the inlet channel 304.

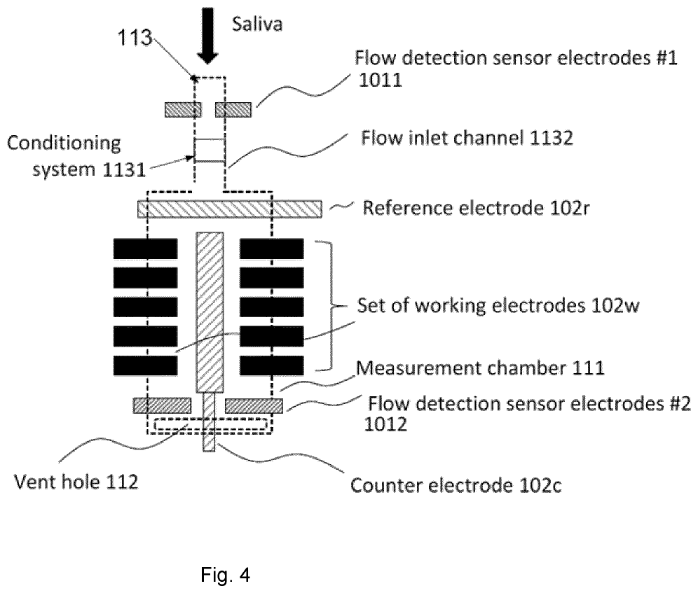

In some embodiments, the caps and/or cartridges may comprise two or more electrodes interconnected by resistors, which may be deposited e.g. by printing or vapour deposition. Such electrodes may be located on a cap surface that is brought into contact with the test strip upon insertion. An electric contact with electrodes in the test strip may then be created upon application, e.g., insertion, of the cap to the test strip. Electrode(s) may be deposited by vapour deposition or by printing (e.g., screen, inkjet, gravure) on the base layer and or on the top layer of the test strip. These may be cap detect (for cap presence detection) electrodes and/or biosensing electrodes, and/or may be couple-able to a reader apparatus/device by electrical interface(s) 105 as shown in FIG. 1. An example layout of such electrodes is shown in FIG. 4, wherein there may be present any one of more of: cap detection electrodes; first and second flow detection sensor electrodes 1011, 1012; reference electrode(s) 102r; working electrode(s) 102w; and/or counter electrode(s) 102c. Such electrodes may generally be combined with the saliva flow inlet channel 1132 of the saliva receiver 113 comprising the conditioning system 1131; measurement or test chamber(s) 111; and/or vent hole(s) 112.

Specifically regarding the biosensing electrodes, a set of electrodes comprising at least a working (WE) 102w and a counter electrode (CE) 102c, but also optionally comprising a third electrode that acts as a potential reference electrode (RE) 102r, may be present. An electrochemical measurement for biosensing test may comprise any of cyclic voltammetry, square wave voltammetry, chronoamperometry or other impedimetric measurements.

The incubation chamber may be functionalized with bioreceptors 103—such as antibodies, aptamers and/or peptides—which can selectively bind to an analyte in saliva. The bioreceptors may enable an immunosensing mechanism, for example for the detection of hormone(s) such as cortisol, testosterone, progesterone, oestradiol etc. or the detection of a drug of abuse.

In a preferred implementation the bioreceptors are antibodies, for example hormone-binding antibodies. The antibodies may be attached onto the substrate layer surface, e.g., by functionalisation of PMMA. In another implementation the bioreceptors are aptamers capable of selectively binding to biomarkers in saliva, including hormones, or of selectively binding to a drug of abuse in saliva. For example aptamers may be hormone-binding aptamers. The 5′ end may be modified with a thiol group, for covalent attachment to a gold surface. Other methods of attachment to the gold and/or to the PMMA surface should be included: for example aptamers could be immobilised via a linker molecule using carboxyl-amine conjugation.

A direct saliva sampling embodiment may provide any one or more of the following: dried reagents for dissolution into the saliva; saliva pre-conditioning; electrochemical assay; electrodes for caps insertion detection; laminar capillary flow; multiple sensors integrated on test strip; specific biomarkers, e.g., C, T, sIgA; and/or improved user experience (UX) of sampling, e.g., using flavours.

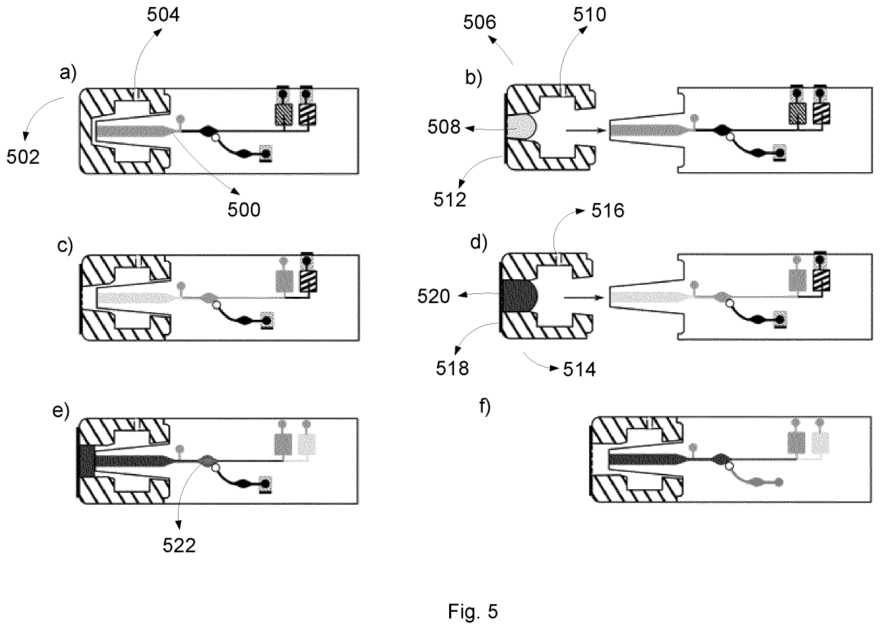

FIGS. 5a-f show exemplary steps, any one or more of which may be used for utilising the test strip 200 in order to allow carry out a competitive assay such as ELISA/ELONA. The diagrams show the saliva sample 500, the wash buffer 508, the substrate solution 520 and the incubated substrate solution 522. A process comprising collecting saliva and/or inserting other solutions into the test strip by opening a sequence of vent holes may be described as follows.

In FIG. 5a, the user has collected the saliva sample 500, and the inlet channel is filled with saliva up to the first (open) vent hole 210. The dried reagents (for example, an analyte-enzyme conjugate such as HRP-estradiol) in the inlet channel dissolve and may mix with the saliva 500 via diffusion. A cap 502 may be placed over the opening of the inlet channel to reduce loss of the saliva sample 500 due to evaporation. The cap 502 may also include a cap vent 504 to prevent overpressure in the cap once the test strip is applied/inserted. Mixing (as for any other mixing mentioned herein) may occur naturally, and/or with the assistance of mixing actuators.

In an alternative implementation, the first vent hole 210 may be located downstream from the incubation chamber 206. The saliva 500 may then fill the incubation chamber 206 on introduction. In this case the dried reagent may be located proximal to the incubation chamber 206 for resuspension.

In FIG. 5b, the cap may be removed and the test strip may be applied/inserted into a first cartridge 506 preferably containing a wash buffer solution 508. The first cartridge 506 may include one or more cartridge vents 510 to reduce/prevent overpressure. This cartridge 506 may be formed of hydrophobic materials, and/or internally coated in a hydrophobic layer. This coating, combined with a hydrophilic film 512, may maintain the wash buffer 508 as a drop on the hydrophilic film 512. Any and/or all cartridges may similarly reduce/prevent evaporation of the saliva sample and/or substance(s) such as reagent(s) while attached to the test strip 200.

In FIG. 5c the second vent hole 212 is opened preferably by removing or piercing the tape covering the vent hole. Opening this vent hole 212 may allow the saliva and mixed reagents to flow into the incubation chamber 206 and first microfluidic channel 214. In this embodiment, such flow may be due to capillary action in the first microfluidic channel 214 and/or the narrowing (constricted section) 204 of the inlet channel. The wash buffer simultaneously flows into the inlet channel, occupying the volume vacated by the flow of the saliva. In the incubation chamber 206 antibodies may be functionalised on the microfluidic substrate, and there may be a competitive reaction for antibody binding sites between free analytes and the reagent. In this example, antibody binding sites may bind to free estradiol in the saliva and estradiol components of the HRP-estradiol conjugates. The antibody surface concentrations may be optimised according to the assay requirements.

In FIG. 5d the test strip 200 is removed from the first cartridge 506 and may be inserted into the second cartridge 514. This second cartridge 514 may largely have the same structure of as the first cartridge 506, for example including a hydrophilic film 518 and/or a vent hole 516, and/or being made from a hydrophobic material and/or internally coated in a hydrophobic layer. However, the second cartridge 514 may contain a substrate solution 520 in place of the wash buffer 508. It is not necessary that the substrate solution 520 and wash buffer 508 have equal volumes. The required volume of each may vary with the chosen substrate solution 520 and/or the dimensions of the test strip 200. In this example, the substrate solution 520 may comprises a TMB and/or H₂O₂ solution, and/or may have a greater volume than the wash buffer 508.

In FIG. 5e, the third vent 216 is opened preferably by piercing or removing the sealing tape. This may allow the saliva solution 500 and/or wash buffer 508 to flow into the second microfluidic channel 218; again this may be due to capillary action. This may allow the substrate solution 520 to flow into the incubation chamber 206. The incubation of the substrate solution involves the substrate reacting with bound reagents in the incubation chamber. For example, a TMB substrate solution reacts with the HRP enzyme of bound HRP-estradiol conjugates.

In FIG. 5f, a fourth vent 224 may be opened preferably by piercing or removing the tape. This may allow the incubated (e.g., TMB) substrate 522 to flow to the test chamber 208 for example via capillary action, wherein the biosensing test may be performed.

It should be noted that the above steps and architecture of the test strip 200 are merely exemplary, and depending on the biomarker or drug of abuse being detected and/or the assay performed a different number, order and/or type of reagents may be required. In some embodiments the reagent may be in the form of a liquid enzyme conjugate solution, rather than a dry enzyme conjugate. In such an embodiment, the liquid reagent may be mixed with the saliva sample after flowing out of an internal reservoir and/or being introduced into the test strip from one or more cartridge(s).

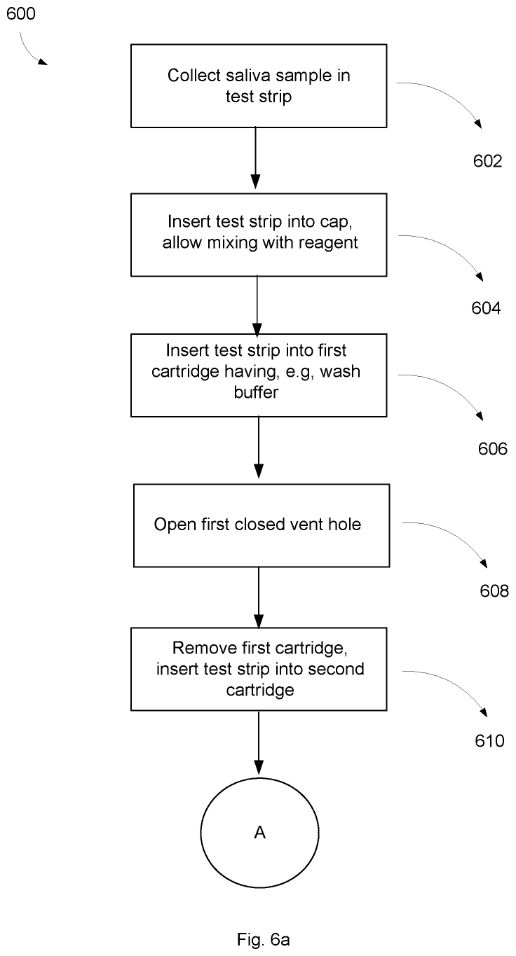

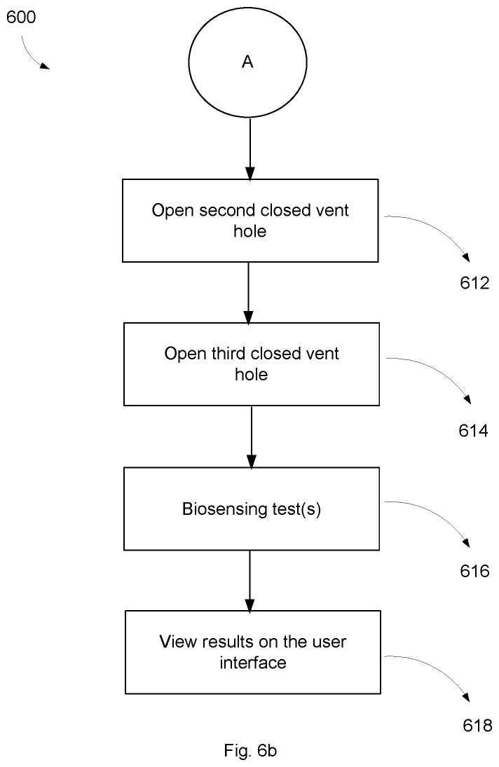

FIGS. 6a and 6b show a flow chart 600 indicating steps carried out by a user during the above exemplary process. Again depending on the particular assay requirements, one or more of the steps may be unnecessary, and/or additional steps may be performed. At step 602, the user collects a sample of saliva preferably in a test strip 200. This may involve holding the test strip in the user's mouth for a specified period of time, or otherwise allowing the inlet channel to fill. At step 604, the saliva may mix with dried reagents (for example, an analyte-enzyme conjugate) preferably coating the inlet channel. In order to reduce/prevent evaporation of the sample during this period, the user may insert the test strip into the cap 502. The user may leave the cap on the test strip until a period sufficient for the saliva and dried reagents to mix has passed. As with all periods discussed herein, the length of this period may be dependent upon the requirements of the assay being utilised. At step 606, the test strip, preferably removed from the cap, may be inserted into a first cartridge 506. The first cartridge may contain the wash buffer 508. At step 608, the first closed vent hole 212 may be opened, allowing the fluids in the test strip to flow down microfluidic channel 214. This step may occur automatically in response to the detection of the cartridge by detection electrodes. Opening this vent hole may allow the saliva sample to flow into the incubation chamber 206. The user may leave the test strip in this state until a specified period of time has passed, in order to allow sufficient time for the competitive reaction for antibody binding sites between saliva analytes and the analyte-enzyme conjugates. At step 610, the user may insert the test strip into the second cartridge 514, preferably after separating the test strip and first cartridge. The second cartridge may contain the substrate solution 520. At step 612, the user may open the second closed vent hole 216; this may allow the fluid in the test strip to flow down microfluidic channel 218. Once again, in some embodiments, this step may occur automatically in response to the detection of the cartridge by the cap detection electrodes. The test strip architecture may be designed such that the substrate solution flows into the incubation chamber following the opening of the second closed vent. In this case, the user may allow the substrate solution to incubate in the incubation chamber for a specified period of time, so as to allow the substrate to react with the bound conjugate. At step 614, the user may open the third closed vent hole 224; this may allow the incubated substrate solution to flow into the test chamber 208. At step 616 a voltage or current may be applied to the biosensing test electrodes. Finally, at step 618 the user may view the results on a UI.

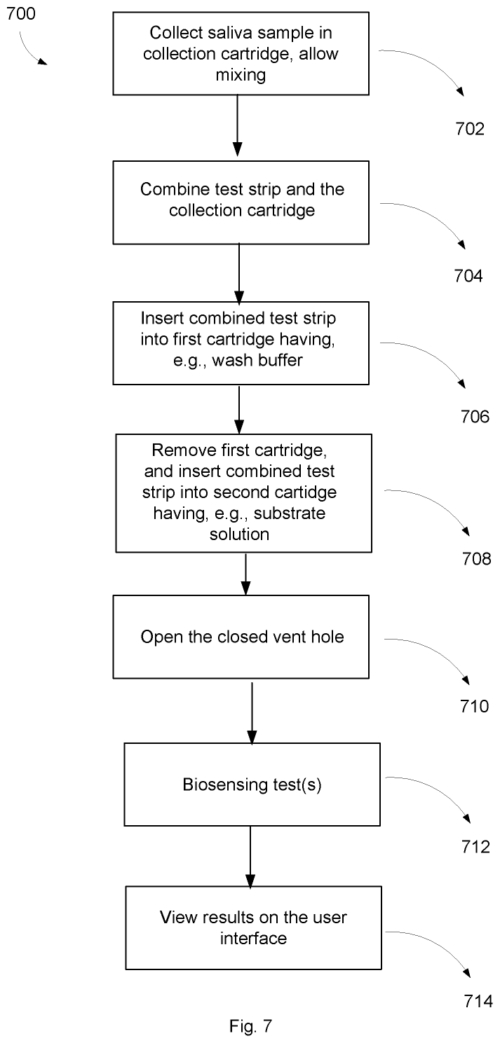

Similarly, FIG. 7 is a flow chart 700 indicating steps (any one or more of which are optional) carried out by the user when carrying out an example assay with the example test strip 300. At step 702, the user collects a sample of saliva in the saliva collection cartridge 302. This collection may involve holding the cartridge in the user's mouth for a specified period of time, or otherwise allowing the inlet channel to fill. After collecting the sample, the saliva may mix with dried reagents coating the collection chamber 322. During this mixing process, the cartridge may be inserted into a cap to prevent or reduce evaporation of the saliva. At step 704, during or after allowing time for the saliva to mix with the reagents in the collection chamber, the user may insert test strip 300 into the collection cartridge, e.g., by breaking the seal 330, and forming the combined test strip. The saliva sample, with mixed reagents, may flow into the incubation chamber 308. At step 706, the combined test strip may be inserted into a first cartridge containing a wash buffer solution, preferably after removing the cap. In some embodiments, the saliva collection cartridge 302 may be separated from the test strip 300 at this stage, and the test strip 300 may be directly applied to, e.g., inserted into, the first cartridge. It is further noted that, in some embodiments, the order of steps 704 and 706 may be reversed; the saliva collection cartridge 302 being inserted into the first cartridge prior to, during or after forming the combined test strip. At step 708, the combined test strip may be inserted into the second cartridge containing a substrate solution, preferably removing the first cartridge. However, the saliva collection cartridge 302 may be, or have been, removed during a previous step, and the test strip 300 may be inserted directly into the second cartridge. At step 710, the closed vent hole 316 may be opened; this may allow the substrate solution to flow into the test chamber 314. At step 712 a voltage or current may be applied to the biosensing test electrodes. Finally, at step 714 the user may view the results on a UI.

Further regarding FIGS. 6 and 7, different assays may require fewer or additional steps, or require that these steps be performed in different orders (e.g., some steps may be performed in reverse or in parallel). For some assays that require longer incubation times, additional closed vent holes may be included, connected to the example architecture so as to control the flow of the substrate solution from the incubation chamber to the test chamber.

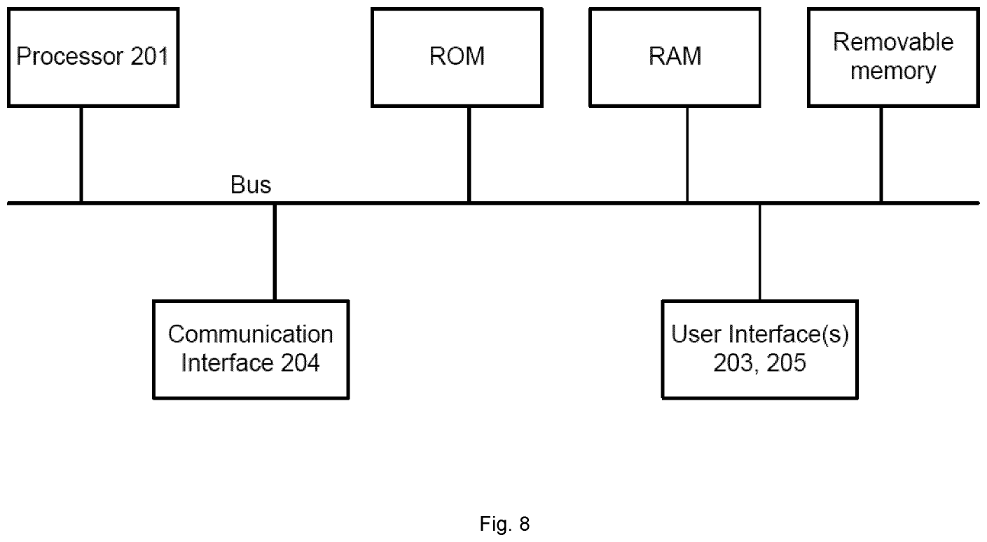

FIG. 8 shows an example computing device or system on which an embodiment, including any element of a saliva test system, such as a test strip, reader apparatus/device, remote and/or mobile computing device as described herein, may be implemented. The computing device/system such as that of FIG. 8 comprises a bus, a controller 201 in the form of at least one processor, at least one communication port (e.g., RS232, Ethernet, USB, etc.) preferably in the form of a wireless interface 204, and/or memory, all generally coupled by a bus (e.g., PCI, SCSI). Each element of FIG. 8 is optional. The memory may comprise non-volatile memory such as read only memory (ROM) or a hard disk and/or volatile memory such as random access memory (RAM, e.g., SRAM or DRAM), cache (generally RAM) and/or removable memory (e.g., EEPROM or flash memory). The processor may be any known processor, e.g., an Intel (registered trademark) or ARM (registered trademark) processor. UIs (e.g., the first 203 and/or second UI 205 described herein) may be provided for example as display screen(s), LEDs or other visual and/or audio output device(s) and/or keyboards. The controller or processor 201 may be an ARM (RTM) device or a similar processor produced by another manufacturer such as Intel (RTM).

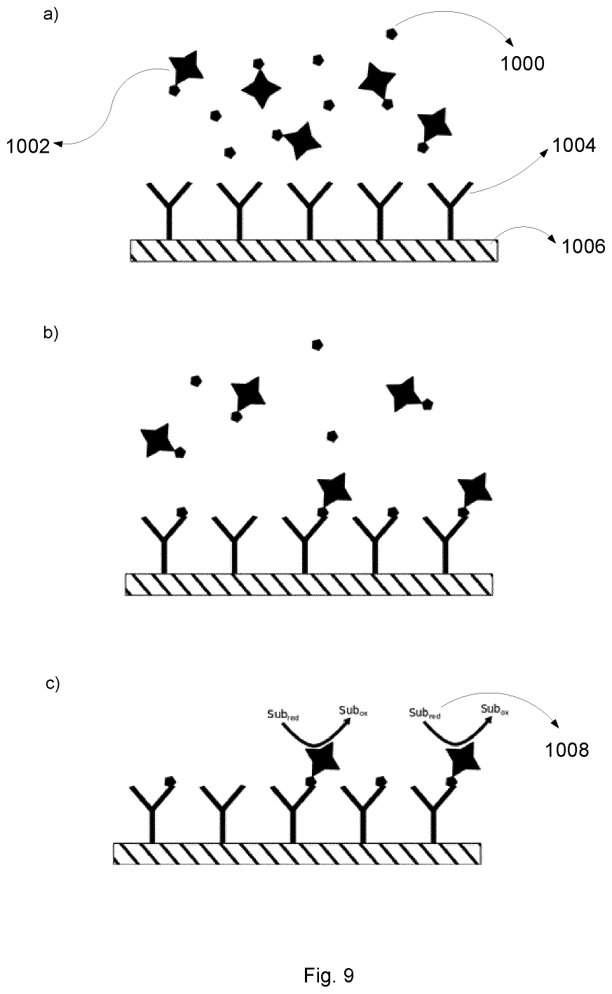

FIGS. 9a)-c) show an example ELISA process that may be performed in an embodiment. In FIG. 9a), a reagent (for example, an analyte-enzyme conjugate) may be resuspended into a saliva sample by mixing. The reagent may be initially suspended in a dry-down buffer, which may include excipients such as salts, sugars, and/or other species such as FeSO4-EDTA that may have been shown to maximise the activity of the enzyme after dry storage. The amount of conjugate dried down may depend on several factors including the diffusion coefficient of the analyte-enzyme conjugate 1002 relative to the free analytes 1000, and/or the relative affinity of the antibody to the conjugate 1002 versus the free analyte 1000. The antibody affinity is preferably greater for the free analyte 1000 and the conjugate 1002 may have a lower diffusion coefficient; in embodiments, this may require the conjugate concentration to be greater than the upper bound of the dynamic range of the assay.

In embodiments, for a dynamic range of 1 pM to 100 pM and a sample saliva volume of 10 μL, the amount of dried down conjugate may be in the range of about 1 fmol to about 1000 fmol.

In FIG. 9b), the analytes 1000 and conjugates 1002 may bind to the bioreceptors 1004, for example antibodies. The antibodies may be functionalised on the microfluidic substrate 1006. Various attachment chemistries may be suitable for such functionalisation, e.g., the use of a biotinylated antibody attached to a streptavidin coating, attachment of antibody to protein-G coating, and/or EDC-NHS chemistry. The analytes 1000 and conjugates 1002 may compete for the antibody binding sites in a competitive reaction. The antibody surface concentrations may be optimised according to the assay requirements. As illustrated in FIG. 9c), the bound conjugate may react with a substrate solution 1008.

In a preferred embodiment, any one or more (preferably all) of the process steps depicted in FIGS. 9a)-c) occur in at least one the incubation chamber of an embodiment. After reacting with the bound conjugates, the substrate solution may be subjected to a biosensing test in order to detect, or quantify the amount of, the bound conjugate.

No doubt many other effective alternatives will occur to the skilled person. It will be understood that the invention is not limited to the above detailed embodiments.

1. A saliva test system for performing an ELISA or ELONA test, the system comprising:
a saliva receiver having an inlet channel for collecting from a user saliva comprising analyte, the saliva receiver to guide the collected saliva to an incubation chamber;
the incubation chamber having bioreceptors for binding to the analyte and reagent, and for providing an incubated solution;
the incubation chamber to incubate a substrate, the incubation to allow the substrate to react with a said bound reagent and thereby provide the incubated solution;
a test chamber arranged to receive a said incubated solution from the incubation chamber, the test chamber having biosensing test electrodes to perform on the incubated solution a biosensing test;
a controller to control the biosensing test electrodes to perform the biosensing test; and
a user interface to indicate a status of a user based on a result of the biosensing test indicating presence or concentration of the analyte in the collected saliva, wherein the system comprises:
a narrowing, such as a constriction, of the inlet channel, the narrowing for limiting a volume of the collected saliva; and
a vent hole to assist flow of the collected saliva through the saliva receiver toward said incubation chamber, the vent hole coupled downstream of the narrowing and/or the incubation chamber and openable to enable flow of the collected saliva through the narrowing toward the incubation chamber.

2. The system of claim 1, wherein the narrowing has at least one of:
a length of about 1 mm to about 30 mm;
a width of about 0.05 mm to about 1 mm; and
a depth of about 0.1 mm to about 0.7 mm.

3. The system of claim 1 or 2, wherein at least one of:
the reagent has conjugates comprising a conjugated portion comprising the analyte and another conjugated portion, preferably wherein:
the analyte comprises a hormone such as cortisol, testosterone, progesterone or estradiol or the analyte comprises a drug of abuse; and/or
the another conjugated portion comprises an enzyme such as horseradish peroxidase enzyme; and/or
the substrate comprises tetramethylbenzidine; and/or
the incubation chamber for receiving a wash buffer to reduce reaction of the substrate and the reagent.

4. The system of claim 1, comprising at least one additional vent hole and microfluidic channel, said additional vent hole coupled by said microfluidic channel to at least one of the incubation chamber and said test chamber.

5. The system of claim 1, comprising at least one seal movable to open and/or seal a said vent hole to thereby reduce or inhibit a flow, the flow preferably comprising the collected saliva, a reagent solution comprising the reagent, the wash buffer and/or the substrate solution.

6. The system of claim 1, comprising a capillary pump coupled to pump the incubated solution toward the test chamber, the system preferably comprising a microfluidic channel coupled to guide the incubated solution toward the capillary pump.

7. The system of claim 1, wherein at least one of the saliva receiver and the incubation chamber is for combining, such as mixing, of the collected saliva with the reagent to form a solution for the binding of the analyte and reagent to the bioreceptors in the incubation chamber.

8. The system of claim 1, wherein the reagent comprises a dry reagent disposed on a surface of said incubation chamber.

9. The system of claim 1, wherein a said reagent comprises a dry reagent disposed on a surface of the saliva receiver to combine with the collected saliva, the surface preferably of a channel such as the inlet channel.

10. The system of claim 1, wherein the reagent comprises a dry reagent for forming a reagent solution when combined with the collected saliva.

11. The system of claim 1, comprising a cap attachable to the saliva receiver to reduce evaporation of the collected saliva, the cap preferably having a vent hole to reduce any increase of pressure in the cap and/or saliva receiver when the cap is being attached to seal an external opening of the inlet channel.

12. The system of claim 1, comprising at least one cartridge to contain at least one substance, the cartridge attachable to deliver the or each substance to the saliva receiver, wherein the substances comprise the saliva, the reagent, a wash buffer and/or the substrate.

13. The system of claim 12, wherein a said cartridge has an inlet for receiving saliva from a user, the cartridge attachable to deliver the received saliva to the inlet channel of the saliva receiver.

14. The system of claim 13, wherein the saliva receiver comprises at least one said cartridge and the inlet of the cartridge comprises the inlet channel having the narrowing.

15. The system of claim 12, wherein a said cartridge has a vent hole to reduce any increase of pressure in the cartridge when the cartridge is being coupled to deliver a said substance to the saliva receiver.

16. The system of claim 12, wherein a said substance is a solution and a said cartridge has an external aperture to deliver the substance to the saliva receiver and has a hydrophilic inner surface preferably opposite the aperture.

17. The system of claim 16, wherein a said substance is a solution and a said cartridge comprises a hydrophobic surface to reduce spread of the solution from the hydrophilic inner surface, said hydrophobic surface comprising an internal and/or external surface of the cartridge.

18. The system of claim 11, having a separable component comprising a said cap and/or a said cartridge, the saliva receiver comprising a first detection electrode and the separable component comprising a second detection electrode located to contact the first detection electrode to enable detection of coupling of the saliva receiver to the separable component.

19. The system of claim 1, wherein the bioreceptors comprise aptamers, the aptamers preferably for binding to a said analyte comprising a hormone such as cortisol, testosterone, progesterone or estradiol or comprising a drug of abuse.

20. The system of claim 1, wherein the bioreceptors comprise antibodies, the antibodies preferably for binding to a said analyte comprising a hormone such as cortisol, testosterone, progesterone or estradiol or comprising a drug of abuse.

21. The system of claim 1, wherein at least one said biosensing test comprises:
an electrochemical impedance spectroscopy test;
a cyclic voltammetry test;
a square wave voltammetry test; and/or
a test to detect or determine concentration of at least one of:
cortisol;
testosterone;
aldosterone;
progesterone
estradiol;
alpha-amylase;
CRP;
DHEA;
sIgA;
a drug of abuse.

22. A method of performing an assay using the system of claim 1, the method comprising controlling flow of the collected saliva through the system by opening vent holes in sequence, a said opening preferably comprising breaking or removing a seal.

23. The method of claim 22 wherein the system is defined by at least claim 18, wherein the sequential opening of the vent holes occurs automatically in response to the detection of the coupling of the saliva receiver to a said separable component by the detection electrodes.


SEQUENCE LISTING (1 sequence)

SEQ ID NO: 1 - DNA, 75 bp, Artificial Sequence
  estradiol-binding aptamer
atacgagcttgttcaatacgaagggatgccgtttgggcccaagttcggcatagtgtggtgatagtaagagcaatc